(19) United States
(12) Patent Application Publication
(10) Pub. No.: US 2022/0339062 A1
(43) Pub. Date: Oct. 27, 2022

(54) VIBRATING WEARABLE DEVICE

(71) Applicant: XIAMEN ZHI HUI QUAN TECHNOLOGY CO. LTD, Xiamen, Fujian (CN)

(72) Inventors: Ji-Li CHUNG, Xiamen, Fujian (CN); Jia-You HE, Xiamen, Fujian (CN); Yong-Jin HSIAO, Xiamen, Fujian (CN)

(21) Appl. No.: 17/640,200

(22) PCT Filed: Aug. 18, 2020
(86) PCT No.: PCT/CN2020/109770

§ 371 Date: Jul. 15, 2022

(30) Foreign Application Priority Data
Sep. 3, 2019 (CN) 201910827375.8

(51) Int. Cl. A61H 23/02; A61N 5/06

(52) U.S. Cl. CPC A61H 23/02; A61N 5/0613; A61H 2201/165; A61H 2201/5005; A61H 2201/10; A61N 2005/0662; A61N 2005/0645; A61H 2201/0157

(57) ABSTRACT

The present invention relates to a vibrating wearable device including a body. The body includes a first layer and a second layer, wherein a compartment is formed between the first layer and the second layer; the first layer is farther away from the wearer's body surface, but the second layer is closer to the wearer's body surface. Besides, the body includes at least one resonance frequency supply unit disposed within the second layer or inside the compartment according to an arrangement, which can partly or completely cover the body.

This application is a national stage application under 35 U.S.C. § 371 from an international application PCT/CN2020/109770, filed Aug. 18, 2020, which claims the benefit of People's Republic of China application Serial No. 201910827375.8, filed Sep. 3, 2019, the subject matter of which is incorporated herein by reference.

### BACKGROUND OF THE INVENTION

### Field of the Invention

The invention relates in general to a vibrating wearable device, and more particularly to a vibrating wearable device used in bio-resonance therapy.

### Description of the Related Art

Resonance refers to the phenomenon that a physical system vibrates with greater amplitude at specific frequencies than at other frequencies, and these specific frequencies are referred as “resonance frequencies”. At a resonance frequency, a small cyclic vibration is enough to generate a tremendous vibration. Most objects have their fixed physical compositions and structures, and therefore have their own specific resonance frequencies. Most objects are able to accumulate the energy of external fluctuations with resonance frequencies received within a period of time no matter these external fluctuations are in the form of sound, light, mechanical vibration, electric field fluctuations or radio waves. Such external fluctuations are referred as “resonance wave”. Organisms of the same species have specific compositions and structures at various levels, ranging from cells, tissues, organs, to physiological systems. Hence, organisms of the same species have similar frequencies of resonance wave. Generally speaking, natural resonance wave of a human organ is within a frequency range of 3 to 17 Hertz (Hz), and any fluctuations with specific intensity and resonance frequency that act on a specific biological level are referred as “biological resonance wave”.

Bio-resonance therapy applies the principle of resonance, a generally known principle in physics, on an organism. The frequency of the organism is converted by or resonates with the frequency of an external fluctuation. When a suitable amount of physical energy is applied on the human body, tissue cells will generate harmonious resonance, which directly affects brain waves, heart rate, and breathing rhythm of the receiver and cause body cells to generate mild resonance, and make the receiver feel substantiated and soothed.

The implementation of bio-resonance therapy includes various methods for applying some specific frequencies to the body. According to “Bio-resonance Therapy” disclosed in PCT Publication No. WO2012160549, various frequency generation sources of different combinations are imprinted or embedded in individual carriers, which can be worn on a user to achieve the efficacy of bio-resonance therapy. Examples of carriers include ointment, cream, bracelet, necklace and watch.

The Taiwanese Patent Publication No. TW1524880B discloses “Control System of Biological Resonance Wave Acting on Organism and Method Thereof”. Corresponding and variable biological resonance wave is generated according to the physiological information of an organism. The control system applies the biological resonance wave on the organism to regulate or convert the fluctuation frequency of the organism.

According to the somatosensory sound vibrating bed disclosed in the Taiwanese Patent Publication No. TW180375, a vibration unit capable of receiving a low frequency audio signal to generate a vibration is disposed on a water-bed. When receiving a suitable low frequency audio signal, the vibration unit enables the water-bed, which has close contact with the user's body, to transmit somatosensory sound vibration and makes the user's whole body feel lively vibration. At the same time, the user can listen to the music through a speaker arranged near the water-bed or a headset connected to a terminal of the water-bed. Thus, when receiving the somatosensory sound vibration, the user can listen to stereo music, the body and the mind can both be relaxed, and mental stress can be relieved.

In view of the prior art, the applicant of the present invention aims to combine the principle of biological resonance with somatosensory sound vibrating bed to provide the user with a biological resonance frequency, which regulates or converts the user's fluctuation frequency and achieve the efficacy of bio-resonance therapy. However, as the somatosensory sound vibrating bed occupies a large and requires an ample space or a specific venue, the convenience of use is greatly reduced.

To resolve the above problem, the present invention provides a vibrating wearable device that is handy and compact. The vibrating wearable device tightly covers the wearer's body surface to provide the wearer with an accurate specific frequency, which is transmitted to the body or the brain, so as to regulate or convert the fluctuation frequency of the wearer and to provide an effect of biological resonance. The specific frequency can be transmitted in the form of sound, light, far infrared ray, mechanical vibrations, electromagnetic wave, electric currents or any combination thereof. Unlike the somatosensory sound vibrating bed, which requires a higher power, the device of the present invention does not require a high power output and can provide a suitable specific frequency to the wearer through optimization of an ordinary power source. Thus, the wearer, within a short period of time, can feel the specific frequency and stimulation provided by the device of the present invention through the skin, and therefore can quickly receive the efficacy of bio-resonance therapy and restore the original state of coordinated fluctuation frequency.

### SUMMARY OF THE INVENTION

The invention is directed to a vibrating wearable device, and more particularly to a vibrating wearable device used in bio-resonance therapy.

To achieve the above objects, the present invention provides a vibrating wearable device including a body. The body includes a first layer and a second layer, wherein a compartment is formed between the first layer and the second layer; the first layer is farther away from the wearer's body surface, but the second layer is closer to the wearer's body surface. Besides, the body includes at least one resonance frequency supply unit disposed within the second layer or inside the compartment according to an arrangement, which can partly or completely cover the body.

As described above, the frequency supply unit of the vibrating wearable device includes at least one frequency generation unit, which can generate at least one specific frequency.

As described above, the vibrating wearable device further includes a control unit electrically connected to the frequency generation unit. The control unit can emit a signal to control or adjust the specific frequency generated by the frequency generation unit to frequency or waveform with the same phase.

As described above, the specific frequency generated by the frequency generation unit of the vibrating wearable device can be directly or indirectly transmitted to the wearer's body surface. The form of the specific frequency includes but is not limited to one or any combinations of sound, light, far infrared ray, mechanical vibration, electromagnetic wave and alternating current.

As described above, the specific frequency of the vibrating wearable device has a frequency range of 1 to 100000 Hertz (Hz).

As described above, the specific frequency of the vibrating wearable device has a preferred frequency range of 3 to 1000 Hz.

As described above, the specific frequency of the vibrating wearable device has a superior frequency range of 5 to 160 Hz.

As described above, the specific frequency of the vibrating wearable device can stimulate the fluctuation frequency of a part or the entirety of the wear's body to achieve resonance.

As described above, the vibrating wearable device further includes a light source outputting element disposed within the wearer's visual range. The light source outputting element can output a visible light according to the resonance frequency to provide stimulation to the wearer's sensory organs and enhance the effect acted on the wearer by the specific frequency.

As described above, the second layer of the vibrating wearable device a composite structure layer capable of transmitting vibrations.

As described above, the vibrating wearable device includes one or any combinations of a hat, clothes, pants, gloves, socks, shoe covers, and a spine back corset posture corrector.

As described above, the arrangement of the frequency supply unit of the vibrating wearable device at least covers the position of an acupoint on the human body.

As described above, the present invention also provides a vibrating wearable device, wherein the frequency supply unit includes a combination of at least one frequency generation unit and at least one frequency conduction element. The frequency generation unit is physically connected to the frequency conduction element to generate at least one specific frequency transmitted through the frequency conduction element. After the specific frequency transmitted by the frequency conduction element enters the compartment, the contact area between the specific frequency and the skin is expanded through the vibration of the compartment cavity.

As described above, the vibrating wearable device further includes a control unit electrically connected to the frequency generation unit. The control unit can emit a signal to control or adjust the specific frequency generated by the frequency generation unit to frequency or waveform with the same phase.

As described above, the specific frequency generated by the frequency generation unit of the vibrating wearable device can be directly or indirectly transmitted to the wearer's body surface. The form of the specific frequency includes one or any combinations of sound, light, far infrared, mechanical vibration, electromagnetic wave, and alternating current.

As described above, the specific frequency of the vibrating wearable device has a frequency range of 1 to 100000 Hz.

As described above, the specific frequency of the vibrating wearable device has a preferred frequency range of 3 to 1000 Hz.

As described above, the specific frequency of the vibrating wearable device has a preferred frequency range of 5 to 160 Hz.

As described above, the specific frequency of the vibrating wearable device can stimulate the fluctuation frequency of a part or the entirety of the wear's body to achieve resonance.

As described above, the vibrating wearable device further includes a light source outputting element disposed within the wearer's visual range. The light source outputting element can output a visible light according to the resonance frequency to provide stimulation to the wearer's sensory organs and enhance the effect acted on the wearer by the specific frequency.

As described above, the second layer of the vibrating wearable device is a composite structure layer capable of transmitting vibrations.

As described above, the vibrating wearable device includes one or any combinations of a hat, clothes, pants, gloves, socks, shoe covers, and a spine back corset posture corrector.

As described above, the arrangement of the frequency supply unit of the vibrating wearable device at least covers the position of an acupoint on the human body.

According to the above description, the present invention can achieve the following technical effects:

1. In one embodiment of the present invention, the venue where the wearer can use the vibrating wearable device of the present invention is not limited to an ample space or a specific venue, so the convenience of use is greatly increased.

2. In one embodiment of the present invention, the wearer of the vibrating wearable device can experience the resonance frequency and stimulation provided by the vibrating wearable device within a short period of time through a larger area of body surface, and can quickly achieve the efficacy of bio-resonance therapy and restore the original state of coordinated fluctuation frequency.

### BRIEF DESCRIPTION OF THE DRAWINGS

FIG. 1 is an overall structural diagram of a vibrating wearable device according to an embodiment of the present invention.

FIG. 2 is a side cross-sectional view of a vibrating wearable device according to another embodiment of the present invention.

FIG. 3 is an overall structural diagram of a vibrating wearable device according to another embodiment of the present invention.

FIG. 4 is an overall structural diagram of a vibrating wearable device according to another embodiment of the present invention.

### DETAILED DESCRIPTION OF THE INVENTION

Detailed descriptions of the technical contents, features and effects of the present invention are described below with accompanying drawings and a number of preferred but non-limiting embodiment(s). The accompanying drawings of the present invention merely serve to make the specification better understood, and are not necessarily based on actual proportions or accurate disposition of the implementation of the present invention. Therefore, the scope of protection of the present invention shall not be limited by the interpretation of dimension scales and disposition relationships in the accompanying drawings.

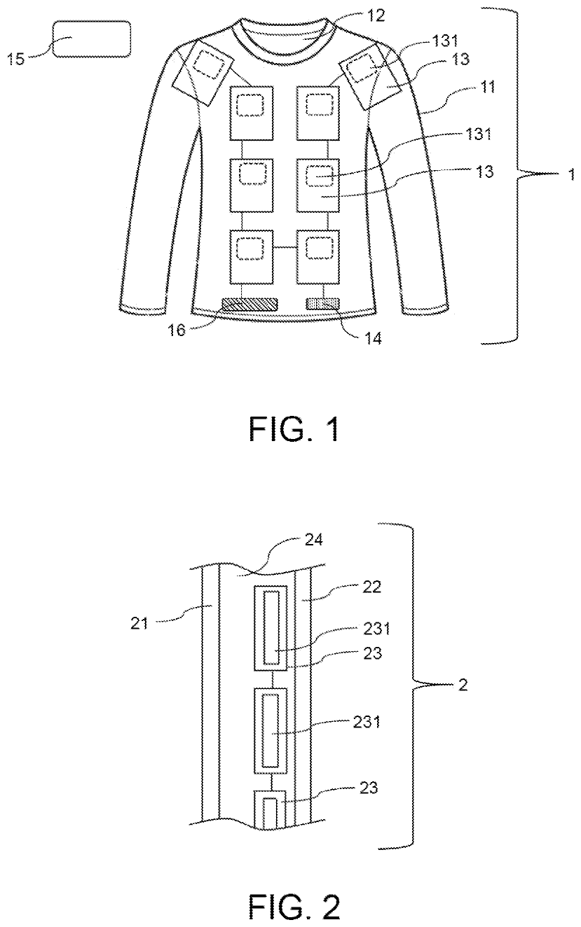

Referring to FIG. 1, an overall structural diagram of a vibrating wearable device according to an embodiment of the present invention is shown. The vibrating wearable device includes a body 1. The body 1 includes a first layer 11 and a second layer 12, wherein a compartment is formed between the first layer 11 and the second layer 12; the first layer 11 is farther away from the wearer's body surface, but the second layer 12 is closer to the wearer's body surface. Additionally, the body 1 includes at least one frequency supply unit 13 disposed within the second layer 12 or inside the compartment according to an arrangement, which can partly or completely cover the body 1. The frequency supply unit 13 further includes at least one frequency generation unit 131. The body 1 further includes a power source 16 for providing power to the frequency generation unit 131.

The vibrating wearable device of the present embodiment further includes a control unit 14 electrically connected to the frequency generation unit 131. The control unit 14 can emit a signal to control or adjust the specific frequency generated by the frequency generation unit 131 to frequency or waveform with the same phase to achieve an optimal effect of resonance. The specific frequency generated by the frequency generation unit 131 can be directly or indirectly transmitted to the wearer's body surface. The form of the resonance frequency includes one or any combinations of sound, light, far infrared ray, mechanical vibration, electromagnetic wave and alternating current. The specific frequency has a frequency range of 1 to 10000 Hz, a preferred frequency range of 3 to 1000 Hz, and a superior frequency range of 5 to 160 Hz.

The vibrating wearable device of the present embodiment further includes a light source outputting element 15 disposed within the wearer's visual range. The light source outputting element 15 can output a visible light according to the specific frequency to provide stimulation to the wearer's sensory organs and enhance the effect acted on the wearer by the specific frequency. The visual range preferably is less than 5 m and superiorly is less than 30 cm.

The second layer 12 of the vibrating wearable device of the present embodiment is a composite structure layer capable of transmitting vibrations and is formed of one or any combinations of cotton, linen, wool, silk, rayon, nylon, tedlon, acrylic fiber, acetate fiber, triacetate fiber, elastane fiber, glass fiber, metal fiber, rubber fiber, neoprene laminate fabric, PVC film composite fabric, and non-woven fabric.

The arrangement of the frequency supply unit 13 of the vibrating wearable device of the present embodiment at least covers the position of an acupoint on the human body. For example, the arrangement covers the jianyu point (LI-15), the danzhong point (CV-17), the zhongwan point (CV-12), the shenque point (CV-8), the tianshu point (ST-25), the qihai point (CV-6), the zusanli point (ST-36), the yongquan point (KI-1), the mingmen point (GV-4), the huiyin point (GV-1), the baihui point (GV-20), and the sanyinjiao point (SP-6).

The vibrating wearable device of the present embodiment makes the wearer, within a short period of time, feel the specific frequency and stimulation provided by the device of the present invention through the body surface, and therefore can quickly receive the efficacy of bio-resonance therapy and restore the original state of coordinated fluctuation frequency.

Referring to FIG. 2, a side cross-sectional view of a vibrating wearable device according to another embodiment of the present invention is shown. The vibrating wearable device includes a body 2. The body 2 includes a first layer 21 and a second layer 22, wherein a compartment 24 is formed between the first layer 21 and the second layer 22. The frequency supply unit 23 is disposed inside the compartment 24 and is arranged according to an arrangement, which arrangement can partly or completely cover the body 2. The frequency supply unit 23 includes at least one frequency generation unit 231.

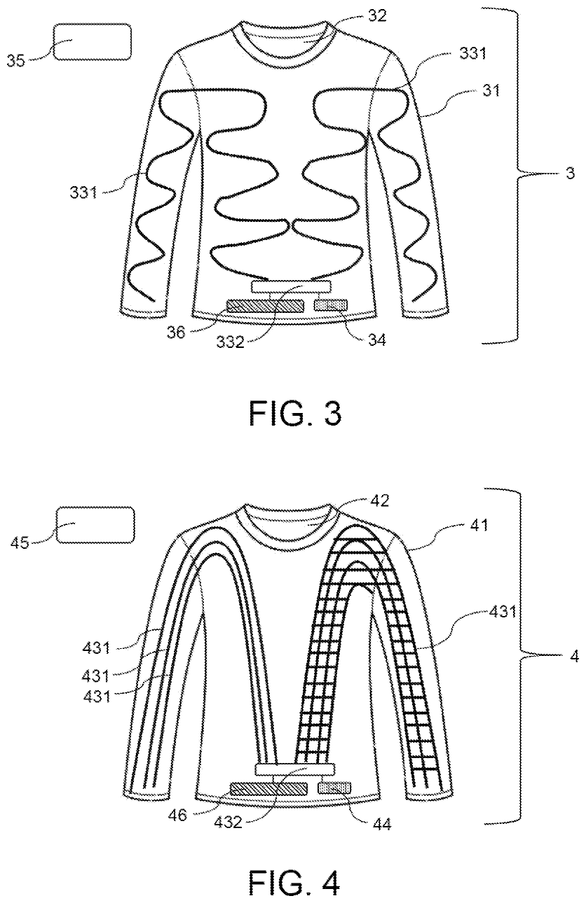

Referring to FIG. 3, an overall structural diagram of a vibrating wearable device according to another embodiment of the present invention is shown. The vibrating wearable device includes a body 3. The body 3 includes a first layer 31 and a second layer 32, wherein a compartment is formed between the first layer 31 and the second layer 32; the first layer 31 is farther away from the wearer's body surface, but the second layer 32 is closer to the wearer's body surface. Additionally, the body 3 includes at least one frequency supply unit (not illustrated) composed of at least one frequency generation unit 332 and at least one frequency conduction element 331. The frequency generation unit 332 is physically connected to the frequency conduction element 331 for generating at least one specific frequency, which can be conducted to the frequency conduction element 331. The frequency supply unit is disposed within the second layer 32 or inside the compartment according to an arrangement, which can partly or completely cover the body 3. The body 3 further includes a power source 36 for providing power to the frequency generation unit 332.

The vibrating wearable device of the present embodiment further includes a control unit 34 electrically connected to the frequency generation unit 332. The control unit 34 can emit a signal to control or adjust the specific frequency generated by the frequency generation unit 332 to frequency or waveform with the same phase to achieve an optimal effect of resonance. The specific frequency generated by the frequency generation unit 332 is then directly or indirectly transmitted to the wear's body by the frequency conduction element 331. The form of the specific frequency includes one or any combinations of sound, light, far infrared ray, mechanical vibration, electromagnetic wave and alternating current. The specific frequency has a frequency range of 1 to 10000 Hz, a preferred frequency range of 3 to 1000 Hz, and a superior range of 5 to 160 Hz.

The vibrating wearable device of the present embodiment further includes a light source outputting element 35 disposed within the wearer's visual range. The light source outputting element 35 can output a visible light according to the specific frequency to provide stimulation to the wearer's sensory organs and enhance the effect acted on the wearer by the specific frequency. The visual range preferably is less than 5 m and superiorly is less than 30 cm.

The second layer 32 of the vibrating wearable device of the present embodiment is a composite structure layer capable of transmitting vibrations and is formed of one or any combinations of cotton, linen, wool, silk, rayon, nylon, tedlon, acrylic fiber, acetate fiber, triacetate fiber, elastane fiber, glass fiber, metal fiber, rubber fiber, neoprene laminate fabric, PVC film composite fabric, and non-woven fabric.

The arrangement of the frequency supply unit of the vibrating wearable device of the present embodiment at least covers the position of an acupoint on the human body. For example, the arrangement covers the jianyu point (LI-15), the danzhong point (CV-17), the zhongwan point (CV-12), the shenque point (CV-8), the tianshu point (ST-25), the qihai point (CV-6), the zusanli point (ST-36), the yongquan point (KI-1), the mingmen point (GV-4), the huiyin point (GV-1), the baihui point (GV-20), and the sanyinjiao point (SP-6).

The vibrating wearable device of the present embodiment makes the wearer, within a short period of time, feel the specific frequency and stimulation provided by the device of the present invention through the body surface, and therefore can quickly receive the efficacy of bio-resonance therapy and restore the original state of coordinated fluctuation frequency.

Referring to FIG. 4, an overall structural diagram of a vibrating wearable device according to another embodiment of the present invention is shown. The vibrating wearable device includes a body 4. The body 4 includes a first layer 41 and a second layer 42, wherein a compartment is formed between the first layer 41 and the second layer 42; the first layer 41 is farther away from the wearer's body surface, but the second layer 42 is closer to the wearer's body surface. Additionally, the body 4 includes at least one frequency supply unit (not illustrated) composed of at least one frequency generation unit 432 and at least one frequency conduction element 431. The frequency generation unit 432 is physically connected to the frequency conduction element 431 for generating at least one specific frequency, which can be conducted to the frequency conduction element 431. The frequency supply unit is disposed within the second layer 42 or inside the compartment according to an arrangement, which can partly or completely cover the body 4. The body 4 further includes a power source 46 for providing power to the frequency generation unit 432.

The vibrating wearable device of the present embodiment further includes a control unit 44 electrically connected to the frequency generation unit 432. The control unit 44 can emit a signal to control or adjust the specific frequency generated by the frequency generation unit 432 to frequency or waveform with the same phase to achieve an optimal effect of resonance. The specific frequency generated by the frequency generation unit 432 is then directly or indirectly transmitted to the wear's body by the frequency conduction element 431. The form of the specific frequency includes one or any combinations of sound, light, far infrared ray, mechanical vibration, electromagnetic wave and alternating current. The specific frequency has a frequency range of 1 to 10000 Hz, a preferred frequency range of 3 to 1000 Hz, and a superior range of 5 to 160 Hz.

The vibrating wearable device of the present embodiment further includes a light source outputting element 45. The light source outputting element 45 has a blindfold design which can be directly used on the wearer's eyes. The light source outputting element 45 can emit a visible light to provide stimulation to the wearer's sensory organs and enhance the effect acted on the wearer by the specific frequency.

The second layer 42 of the vibrating wearable device of the present embodiment is a composite structure layer capable of transmitting vibrations and is formed of one or any combinations of cotton, linen, wool, silk, rayon, nylon, tedlon, acrylic fiber, acetate fiber, triacetate fiber, elastane fiber, glass fiber, metal fiber, rubber fiber, neoprene laminate fabric, PVC film composite fabric, and non-woven fabric.

The arrangement of the frequency supply unit of the vibrating wearable device of the present embodiment at least covers the position of an acupoint on the human body. For example, the arrangement covers the jianyu point (LI-15), the danzhong point (CV-17), the zhongwan point (CV-12), the shenque point (CV-8), the tianshu point (ST-25), the qihai point (CV-6), the zusanli point (ST-36), the yongquan point (KI-1), the mingmen point (GV-4), the huiyin point (GV-1), the baihui point (GV-20), and the sanyinjiao point (SP-6).

When the user is lying on the somatosensory sound vibrating bed of the prior art, the weight of the body enables the frequency of the same phase generated by the same vibrating source to be transmitted all over the body. However, the same effect cannot be achieved when the user uses a wearable device. To resolve this technical problem, the vibrating wearable device of the present invention further includes a compartment. After specific frequency generated by the frequency supply unit of the present invention is transmitted to the compartment, the contact area between the specific frequency and the skin is expanded through the vibration of the compartment cavity. Thus, the specific frequency can be transmitted to the wear's sense organs to induce the brain to generate corresponding biological resonance, so that the wearer's body and mind can both be regulated.

In the vibrating wearable device of the present invention, the frequency supply unit can include a frequency generation unit only or a combination of a frequency generation unit and a frequency conduction element. The control unit of the vibrating wearable device of the present invention can emit a signal to control or adjust the specific frequency generated by the frequency generation unit, such that each specific frequency generated by the frequency generation unit can remain as frequency or waveform with the same phase. Besides, the frequency conduction element is a solid structure similar to a conducting wire, which possesses the features of smaller contact area and longer transmission distance. Since the frequency generation unit is physically connected to the frequency conduction element, the specific frequency generated by the frequency generation unit can be synchronically conducted to the compartment through the direct transmission performed by the frequency conduction element. After the specific frequency is synchronically conducted to the compartment, the contact area between the specific frequency and the skin is expanded through the vibration of the compartment cavity, and the specific frequency can be transmitted to the wear's sense organs to induce the brain to generate corresponding biological resonance.

The vibrating wearable device of the present embodiment, when used in bio-resonance therapy, makes the wearer, within a short period of time, feel the specific frequency and stimulation provided by the device of the present invention through the skin and quickly receive the efficacy of bio-resonance therapy, so that the wearer's body can quickly achieve the optimal state of coordinated fluctuation of biological resonance wave.

The vibrating wearable device of the present invention can provide optical and audio stimulation to the wearer by adjusting the frequency to low frequency fluctuations within the Schumann wave range (7.8 to 10 Hz) and using the method provided by L. D. Usanova, A. D. Usanova, and A. V. Skripal (“Analysis of Effect of Audiovisual on Parameters of Encephalogram and Rate of Pulsation Wave Propagation in Humans”, Biomedical Engineering, Vol. 46, No. 1, 2012, pp. 25-28) or Helane Wahbeh, Carlo Calabrese, and Heather Zwickey (“Binaural Beat Technology in Humans: A Pilot Study To Assess Psychologic and Physiologic Effect”, The Journal of Alternative and Complementary Medicine, Vol. 13, No. 1, 2007, pp. 25-32). Thus, the wearer's brain wave can be adjusted to the frequency of alpha waves to achieve an optimal effect of mental relaxation.

While the invention has been described by way of example and in terms of the preferred embodiment (s), it is to be understood that the invention is not limited thereto. Based on the technical features embodiments of the present invention, a person ordinarily skilled in the art will be able to make various modifications and similar arrangements and procedures without breaching the spirit and scope of protection of the invention. Therefore, the scope of protection of the present invention should be accorded with what is defined in the appended claims.

What is claimed is:

1. A vibrating wearable device, comprising:
a body, comprising a first layer and a second layer, wherein a compartment is formed between the first layer and the second layer; the first layer is farther away from a wearer's body surface, and the second layer is closer to the wearer's body surface; and
at least one frequency supply unit disposed within the second layer or inside the compartment according to an arrangement, which partly or completely cover the body.

2. The vibrating wearable device according to claim 1, wherein the frequency supply unit comprises at least one frequency generation unit, which generates at least one specific frequency.

3. The vibrating wearable device according to claim 1, wherein the frequency supply unit comprises a combination of at least one frequency generation unit and at least one frequency conduction element; the frequency generation unit is physically connected to the frequency conduction element to generate at least one specific frequency; after the specific frequency transmitted by the frequency conduction element enters the compartment, the contact area between the specific frequency and the skin is expanded through the vibration of the compartment cavity.

4. The vibrating wearable device according to claim 2, further comprising a control unit electrically connected to the frequency generation unit, wherein the control unit emits a signal to control or adjust the specific frequency generated by the frequency generation unit to frequency or waveform with the same phase.

5. The vibrating wearable device according to claim 2, wherein the specific frequency generated by the frequency generation unit is directly or indirectly transmitted to the wearer's body surface; the form of the specific frequency comprises one or any combinations of sound, light, far infrared, mechanical vibration, electromagnetic wave, and alternating current.

6. The vibrating wearable device according to claim 2, wherein the specific frequency stimulates the fluctuation frequency of a part or the entirety of the wear's body to achieve resonance.

7. The vibrating wearable device according to claim 1, further comprising a light source outputting element disposed within the wearer's visual range, wherein the light source outputting element outputs a visible light according to the resonance frequency to provide stimulation to the wearer's sensory organs and enhance the effect acted on the wearer by the specific frequency.

8. The vibrating wearable device according to claim 1, wherein the second layer is a composite structure layer capable of transmitting vibrations.

9. The vibrating wearable device according to claim 1, comprising one or any combinations of a hat, clothes, pants, gloves, socks, shoe covers, and a spine back corset posture corrector.

10. The vibrating wearable device according to claim 1, wherein the arrangement of the frequency supply unit at least covers the position of an acupoint on the human body.

11. The vibrating wearable device according to claim 3, further comprising a control unit electrically connected to the frequency generation unit, wherein the control unit emits a signal to control or adjust the specific frequency generated by the frequency generation unit to frequency or waveform with the same phase.

12. The vibrating wearable device according to claim 3, wherein the specific frequency generated by the frequency generation unit is directly or indirectly transmitted to the wearer's body surface; the form of the specific frequency comprises one or any combinations of sound, light, far infrared, mechanical vibration, electromagnetic wave, and alternating current.

13. The vibrating wearable device according to claim 3, wherein the specific frequency stimulates the fluctuation frequency of a part or the entirety of the wear's body to achieve resonance.

14. The vibrating wearable device according to claim 2, further comprising a light source outputting element disposed within the wearer's visual range, wherein the light source outputting element outputs a visible light according to the resonance frequency to provide stimulation to the wearer's sensory organs and enhance the effect acted on the wearer by the specific frequency.

15. The vibrating wearable device according to claim 3, further comprising a light source outputting element disposed within the wearer's visual range, wherein the light source outputting element outputs a visible light according to the resonance frequency to provide stimulation to the wearer's sensory organs and enhance the effect acted on the wearer by the specific frequency.
